(19) United States
(12) Patent Application Publication
(10) Pub. No.: US 2014/0301895 A1
(43) Pub. Date: Oct. 9, 2014

(54) IN LINE STERILIZER

(71) Applicants: David B. Opie, Severn Park, MD (US); Evan M. Goulet, Baltimore, MD (US); Blaine G. Doletski, Gatonsville, MD (US); William E. Waters, Millersville, MD (US)

(72) Inventors: David B. Opie, Severn Park, MD (US); Evan M. Goulet, Baltimore, MD (US); Blaine G. Doletski, Gatonsville, MD (US); William E. Waters, Millersville, MD (US)

(73) Assignee: Noxilizer Inc., Baltimore, MD (US)

(21) Appl. No.: 14/237,276

(22) PCT Filed: Aug. 3, 2012
(86) PCT No.: PCT/US12/49610

§ 371 Date: Jun. 25, 2014

Related U.S. Application Data
(60) Provisional application No. 61/515,624, filed on Aug. 5, 2011.

(51) Int. Cl. B65B 55/10

(52) U.S. Cl. CPC B65B 55/10
USPC 422/28; 422/300

(57) ABSTRACT

A sterilization system includes a plurality of modular gas impermeable chambers separable by doors movable between an open position in which gas may flow freely between the adjacent chambers and a closed position in which gas flow between the adjacent chambers is prevented. A conveyor system carries objects between the chambers. Sterilant gas is controllably deliverable to at least one of the chambers. Objects are conveyed between the chambers to execute a sterilization operation.

This application claims priority to U.S. Provisional Patent Application 61/515,624 filed Aug. 5, 2011, which is incorporated by reference in its entirety herein.

### BACKGROUND OF THE INVENTION

1. Field

This application relates generally to sterilization systems and more particularly to sterilization systems for use in in-line processing of packaged products

2. Description of Related Art

In general, ethylene oxide (EtO) systems and/or radiation treatment are used for high volume, batch sterilization of finished goods such as medical devices. Typically, the sites for these processes are located far from the point of manufacture.

US Patent Application 2002/0002912 describes a continuous sterilization and processing system that relies on a pressurized process and steam.

U.S. Pat. No. 7,727,464 describes a sterilization process and transport system that sterilizes the external surface of syringe tubs. A feature of this system is to rapidly evacuate the processing chamber, and then quickly add steam and hydrogen peroxide. The patent teaches that speed of evacuation is important to prevent hydrogen peroxide from penetrating into the interior of the tub, thereby contaminating the tub contents, which may tend to require long aeration times. The chamber is then re-evacuated to remove the sterilant gas.

U.S. Pat. No. 3,761,224 describes a continuous sterilization process where products to be sterilized are moved through a chamber that contains a heavier than air sterilizing gas. The products to be sterilized are conveyed into the chamber, starting at a point above the chamber, proceeding down through a conduit, then after a sufficient period in the chamber, being conveyed up and out of the heavier sterilizing gas, ethylene oxide in the described embodiments. Residual ethylene oxide may remain on surfaces of the treated products, typically requiring an extended aeration period.

### BRIEF SUMMARY OF THE INVENTION

A sterilization system includes a series of chambers, each chamber being configured and arranged to perform a portion of a sterilization process for an object. Objects to be sterilize enter the chambers serially, and undergo respective portions of the sterilization process before being moved to subsequent chambers.

A method of sterilization comprises conveying an object along a conveyor path such that during the conveying steps of the sterilization process are performed.

### BRIEF DESCRIPTION OF THE DRAWINGS

FIG. 1 is a figure illustrating a typical package containing several syringes;

FIG. 2a is a schematic illustration of an in-line sterilizer in accordance with an embodiment;

FIG. 2b shows pressure v. time for a sterilization cycle using an in-line sterilizer as illustrated in FIG. 2a;

FIG. 3a is a schematic illustration of an in-line sterilizer in accordance with an embodiment;

FIG. 3b shows pressure v. time for a sterilization cycle using an in-line sterilizer as illustrated in FIG. 3a; and

FIG. 4 is a schematic illustration of an aeration end of an in-line sterilizer in accordance with an embodiment.

### DETAILED DESCRIPTION OF THE INVENTION

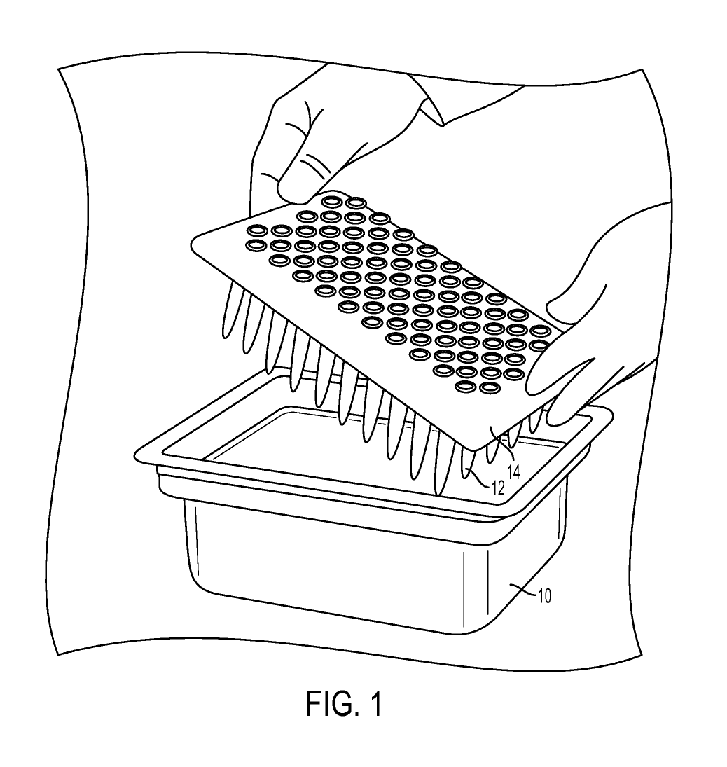

Referring now to FIG. 1, an example of a product or object to be sterilized is shown. A container, or tub, 10 is arranged to hold a tray supporting multiple prefillable syringes 12. In a typical example, the tub may hold 100 syringes that are ready to be filled. The syringes are supported by a plate 14 that includes holes configured to allow a barrel of each syringe to pass through while being supported at an upper end. The plate may be, for example, polypropylene, and the tub may be polystyrene. The tub may include a barrier layer (such as, e.g., Tyvek®) that permits gases to enter and exit the package through this barrier layer and serves as a barrier to microbes and dirt, protecting the tub contents. Similarly, the tub may be sealed with a barrier lid, not shown. Each tub may be placed in a pouch to further protect the tub and syringes.

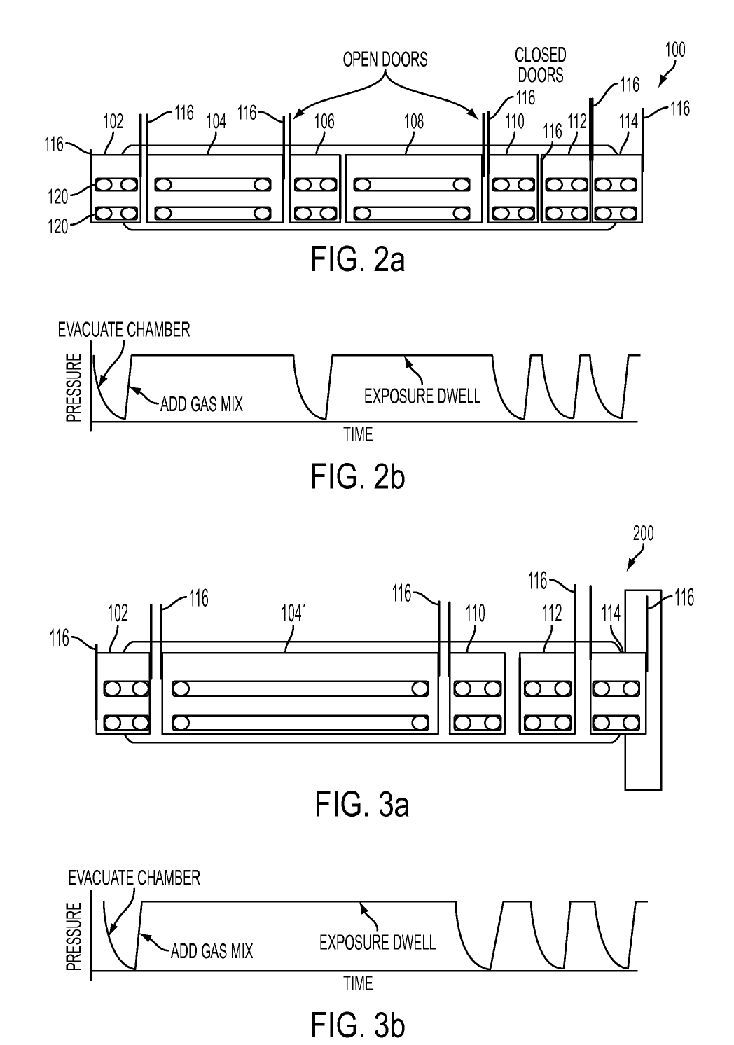

In order to sterilize a tub of this type, a sterilizer 100, as shown in FIG. 2a, may be used. The sterilizer 100 includes several chambers, 102, 104, 106, 108, 110, 112, 114. The chambers are separated by sets of doors 116. In an embodiment as illustrated, each chamber has a door 116 at each end, though in principle a door at only one end of each chamber could be sufficient to separate the chambers. The chambers further include at least one conveyor 120 that conveys objects to be sterilized through the system. In the illustrated embodiment, an upper and a lower conveyor are shown, though the number of conveyors may be selected, for example in accordance with throughput requirements of the system.

The chambers are constructed and arranged to be substantially gas tight when the doors are closed, such that they may be evacuated and/or filled with air, humidified air and/or sterilant gases. The doors may have seals or gaskets made from rubber or other suitable materials. The chambers themselves may be made from metal or plastic, the features of interest being low permeability to gases or fluids, and smooth inner surfaces to discourage adherence or embedding of contaminant particles. In embodiments, hydrophobic or oleophobic coatings may be used to help prevent contaminant adherence.

In embodiments, chambers may include vacuum ports, allowing for evacuation, and/or gas ports, allowing for input of sterilant gas, air, and/or humidity. In this regard there may be multiple gas ports or all gases may be introduced through a common port. Likewise the gases or vacuum lines may pass through a manifold such that a single vacuum source is able to provide vacuum to all of the chambers. In this approach, it may be useful to include a valving system such that individual vacuum lines are separately controllable.

Embodiments may include temperature controls including, for example, temperature sensors, heaters and/or coolers. A humidity sensor may also be included to allow a feedback control of system humidity conditions. In an embodiment, the source of humidity is controlled to provide humidity in vapor form and to avoid delivery of water particles, which may tend to interfere with aspects of the sterilization process.

In an embodiment, one or more chambers may include a radiation system for delivering radiation to the object, either for direct sterilization by radiation, or in a cooperative effect with sterilizing gases. Likewise, one or more chambers may include a system for producing sterilizing gases. For example, a chamber may include an ozone generator, configured to convert oxygen in air, or gaseous oxygen, to ozone for use as a sterilant.

In the example of FIG. 2a, a first chamber 102 that the product enters is configured and arranged to expose products to a sterilizing (or decontaminating) gas mixture. In the cycle illustrated in FIG. 2b, first, the chamber 102 is evacuated, then the sterilant gas is added. The doors 116 are opened to allow the conveyor 120 to pass the object to the second chamber 104. In embodiments, the conveyors are configured such that they extend sufficiently close to the doors that objects conveyed thereon are supported during a transition between adjacent modules. In general, this means that a distance between adjacent conveyors is selected to be less than about half of a length of a base of the objects to be sterilized. Otherwise, the conveying system will have a means to bridge this gap, such as a means that moves into place as the door moves to open the path between adjacent chambers.

In the illustrated example, the second chamber 104 is a dwell chamber where the object is held in exposure at a selected pressure (which may be, for example, ambient pressure, or high or low pressure. In an embodiment, the pressure in the chambers is held as a slight underpressure to reduce the possibility of sterilant gas escaping the system into the ambient environment.

After the dwell operation, the object is passed to a second evacuation chamber 106. In the second evacuation chamber, gases are evacuated, and sterilant gas is added. This chamber then passes the object to a second dwell chamber 108 where a second dwell is performed. As will be appreciated, the time for each dwell may be selected as necessary or desired, and the two dwells need not be of the same duration. After the second dwell, the object is moved to a series of chambers 110, 112, 114 where aeration steps are performed. As can be seen from FIG. 2b, gases are evacuated, then air is delivered to the chamber. In this manner, sterilant gas is removed from the object and its packaging.

As will be appreciated, in the embodiment of FIG. 2a, the two dwell chambers 104 and 108 may omit any connection to gas or vacuum sources, as they may operate strictly as dwell chambers without performing any additions or removal of gases. Alternately, the dwell chambers may include vacuum lines allowing them to be evacuated so that when they are opened to an exposure chamber, the gas from the exposure chamber will tend to fill the dwell chamber.

As described above, to complete each phase of the sterilization process, the product moves by means of conveyor (which may be a conveyor belt or similar mechanism) from one chamber to the next, until the product completes passage through the assembled system. By passing through the complete system, the product is exposed to all phases of the sterilization or decontamination process. In an embodiment, each chamber is the same length, and dwell chambers, for example, are made up of several modules, such that objects may proceed through the system simultaneously, each phase of the cycle corresponding to travel through a single chamber.

As briefly described above, doors 116 that are located between the system chambers limit the uncontrolled movement of gas through the system and allow the evacuation and filling of the different chambers. That is, adjacent chambers may have different pressures, and any atmosphere present within a given chamber may differ from the atmosphere present in its neighbor or neighbors. With the doors closed, the chambers are sealed and a vacuum step can be used to expedite the exchange of gases in the chamber. Thus, any step may include the evacuation of air and the filling of the chamber with sterilant, the removal of sterilant and refilling the chamber with a sterilizing gas mixture, or the removal of sterilant and rinsing the chamber and enclosed product with fresh air, or an inert gas or other gas mixture. For example, where oxidation is a concern, nitrogen gas may be used instead of air.

The opening and closing of the doors is timed to provide an efficient passage of product through the system. The exact sequencing of the doors can be controlled in accordance with the specific goals of a given program or cycle. For embodiments in which there are two doors at each conjunction, the opening and closing of doors may be controlled jointly, so that the two doors open together, or may be independently controlled to allow one chamber to be opened before its neighbor.

In an embodiment, the system is modular. That is, each chamber is configured and arranged to perform a particular sterilization phase, and is further configured and arranged to be modularly connectable to each other chamber. In this approach, a cycle profile can be defined by selection and placement of the various modules. By way of example, dwell time in a particular chamber (and therefore, a particular sterilization phase) can be determined by conveyor speed and/or chamber length. For any selected cycle profile, a set of chambers can be selected to perform the desired operation. In this regard, where a desired cycle consists of evacuation and exposure (Ee), dwell (Dw), Ee, Dw, purge (Pu), Pu, Pu, the chambers may be arranged as illustrated in FIG. 2a. Alternately, to perform a cycle of Ee, Dw, Pu, Pu, Pu, the chambers may be arranged as illustrated in FIG. 3a. Note that the Ee phase of FIG. 3a is for a longer time than the Ee phases of FIG. 2a. Thus, a longer exposure chamber 104′ is used, though alternately, a slower conveyor may be substituted for the longer chamber.

In this approach, because each chamber is dedicated to a particular phase of the process, the cycle profile is simply a function of the placement of the system chambers. A cycle profile may be defined by the pressure and gas compositions to which the object is exposed over time, and may also include temperature and/or humidity dimensions. In accordance with an embodiment, the cycle profile is created by the ensemble of the process phases produced by each chamber in the system. As illustrated in FIGS. 2a and 2b and 3a and 3b, the chamber elements are shown with the corresponding cycle profile to be produced. Therefore, once a process cycle is established for the desired product, the modular elements of the proposed system are assembled to realize the desired cycle.

In an example of an application of the embodiment illustrated in FIG. 2a, newly manufactured products are sterilized in a two exposure process. The first exposure provides the required six-log reduction of a challenge organism population (or other metrics may be applied, depending on the product being sterilized); and, the second exposure phase provides the require sterility assurance level (SAL).

The system configuration shown in FIG. 3a may find application, for example, in completion of surface decontamination of products where two exposure phases are not needed. By way of example, this configuration could be used for the surface decontamination of bulk vials or syringe tubs prior to passing these products into a sterile filling line isolator. Typically, an e-beam system is used for this type of decontamination process. After decontamination, the products would typically enter an aseptic enclosure of a sterile filling line.

As described above, an embodiment involves manufacturing the chambers in a modular fashion, using consistent attachments and interfaces. This may allow for ease of construction of a sterilization system. In one example, the entrance chamber and aeration chambers are approximately 30 inches wide, 12 inches tall, and 12 inches long (in the direction of product travel). These chambers can be built to allow for pressurization and evacuation, and thus should be strong enough to support the external atmospheric pressure when the volume of the chamber is evacuated.

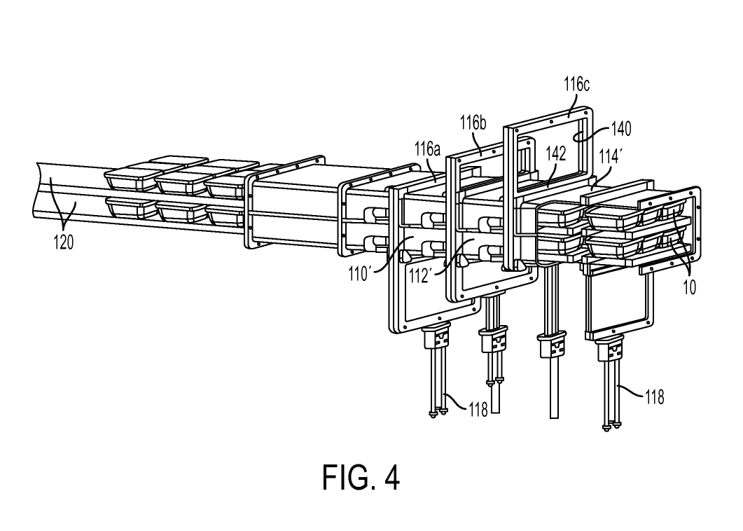

The purge, or aeration, chambers may have the same geometry as the entrance chamber. A schematic illustration of an aeration end of a system in accordance with an embodiment is shown in FIG. 4.

A number of tubs 10 are positioned on conveyors 120. The purge chambers 110′, 112′, 114′ of this embodiment are separated by doors, as in the other illustrations. The doors are movable using actuators 118 to push them upward or pull them downward between open and closed positions.

In the Figure, door 116a is shown in its open position (i.e., gas and/or objects may pass freely into the left side of chamber 110′. Door 116b is partially open and door 116c is in its closed position. Sealing edges 140 of the doors 116a-116c should be configured such that they generally prevent gas flow from either adjacent chamber, whether the door is open, closed, or in between. In this regard, a top sealing edge 142 may remain stationary as the open frame is slid above the top surface of the chamber. In principle, the directions of motion of the doors, and the according selection of stationary sealing members may be altered as necessary or desired.

Generally, the series of chambers may be collectively mounted on a common frame. The frame may also support other components appropriate for each segment of the system.

In an embodiment, the chambers may further include a sensor configured and arranged to verify location and transit of product into and out of the chambers. For example, this may include video cameras, still cameras, light beam/photodiode pairs or the like.

The interfaces that lie between any two chamber elements (the location that includes the chamber doors) should be enclosed to avoid leakage of sterilant into the environment surrounding the system. For example, a shroud/external enclosure panels can be used to accomplish this goal. As noted above, moderate underpressure in the system can assist with controlling any such leakage. Similarly, where an external enclosure is used, an overpressure in the external enclosure may prevent escape of material from the internal modules.

As will be appreciated, the system described may find application with a variety of gaseous sterilants, though the inventors have found particular advantage in use of nitrogen dioxide gas. In use, a sterilization cycle with NO₂ employs between about 5 mg/L to 20 mg/L (roughly 0.25% to 1% at ambient pressure). For tub surface decontamination, 6 mg/L of NO2 (for a total of 6 minutes) is sufficient for achieving the required spore log reduction. The gas delivery may be accomplished by using a DOT approved cylinder holding a quantity of liquid NO₂ (which is actually the dimer N2O4). Nitrogen dioxide boils at room temperature, so that liquid may be used to provide vapor for the chamber without requiring a heating element or other delivery system. In an embodiment, a pre-chamber may be used to generate the appropriate amount of sterilant vapor. A pre-chamber process of this type is described in U.S. patent application Ser. No. 12/710,053, hereby incorporated by reference in its entirety. In another embodiment, a chemical composition that generates NO₂ may be positionable within a sterilizing chamber or in a pre-chamber where it may be activated to generate the NO₂ for sterilization. Alternately, a gas cylinder or other storage device may deliver gas directly.

In embodiments incorporating a vacuum pump for evacuation of the chambers, a scrubber system may be located in the gas circuit between the chambers and the pumps, and used to capture the NO₂. Scrubbers may tend to protect the pump from exposure to sterilant gases, and to avoid release of sterilant from the pump exhaust. In an embodiment, the scrubber system may be configured to reduce the NO₂ concentration in the pump exhaust to <1 ppm. By way of example, exhaust gases may be passed through a permanganate medium to capture the NO₂. Permanganate is a good adsorber of NO₂, and once saturated, is landfill safe. The pumping rate for evacuation pumps may be selected to be sufficient to evacuate the chambers within one minute, or more particularly, within 30 seconds.

A user interface, not shown, may be incorporated allowing for programming of aspects of the system. This may include, for example, timing of stages (i.e., conveyor speed), dosage of sterilant, opening and closing of doors between chambers, humidity and/or temperature, and others. The user interface may also include displays for providing a user with information regarding the defined parameters and/or indications of operating conditions of the system. Controllers can be based on computers, microprocessors, programmable logic controllers (PLC), or the like.

In an example of a use of a device as described above, prefilled syringes are subject to sterilization and/or decontamination at various points in the manufacturing process. A first sterilization process is performed after syringe components are manufactured. A second decontamination (sterilization) process occurs prior to filling the syringes within an aseptic enclosure, often called a filling line. Prior to the syringes entering the filling line, the syringes are exposed to a decontamination process that removes contaminants on the tub surfaces that could compromise the aseptic filling line environment. Finally, after filling, the syringes are removed from the filling line and packaged. In some cases, the filled syringes will be decontaminated or sterilized, as may be needed for the intended application (for example, syringes intended for use in an intraoperative setting).

### EXAMPLE 1

External and internal differences in pressure of prefilled syringes can cause plunger movement during sterilization, which might cause drug product contamination. Consequently the pressure inside the autoclave during sterilization should be controlled carefully to prevent contamination of the drug product by microorganism and particulates. A previously determined theoretical relationship of temperature to pressure in sealed bottles was modified for prefilled syringes to take plunger movement into account. This modification yielded a correction factor that includes a coefficient of linear thermal expansion for the syringe, thermal expansion of the plunger, and friction between the plunger and the syringe wall. To confirm the accuracy of this modified relationship, 100 mL polypropylene prefilled syringes with butyl rubber plungers, some of which carried pressure and temperature sensors, were used to test various sterilization conditions at the experimental scale. The results showed that the major problem in establishing the pressure conditions for production scale sterilization is temperature distribution throughout the load. However, an over pressure sterilization cycle at 121 degrees C. and 0.34 MPa showed the best results. Microbial challenge and light-obscuration particle count tests were performed on the syringes from the worst-case location predicted from modified relationship; the results show that these conditions preserved the sterility of the drug product and protected it from particulate contamination.

### EXAMPLE 2

For both configurations shown in FIGS. 2a and 3a, the first chamber in the system will introduce the sterilant. With products that can tolerate a vacuum phase, then a minimum pressure of 20 mmHg (about 1″ Hg) has been shown to be sufficient for most medical device products. This is not a deep vacuum and can be reached quickly with standard pumps. In an embodiment, six tubs are batch processed in a single chamber (three tubs across the upper conveyor shelf and three tubs across the lower shelf, as shown in FIG. 4). For processing six typical 100 tube tubs per minute, the volume of the chamber would be between 60 liters and 75 liters. A standard pump can evacuate this volume to the target pressure in about 30 seconds. Once this chamber is pumped to the target pressure, this volume is filled with sterilant and humidified air to match the pressure and sterilant concentration of the next chamber.

The second chamber is the exposure chamber, in which the product will remain for the desired exposure duration. It is not necessary for this chamber to be evacuated and filled, as would occur with a traditional sterilization chamber. Rather, this chamber remains at a constant pressure, presenting a consistent exposure condition for the products that traverse this chamber. For products that are exposed for five minutes, and where products enter and exit the system at one minute increments, then this chamber must have a length equivalent to at least five product lengths. For a typical tub, such as the BD Hypak SCF tub, which have a footprint of 8.75 inches long (and 10.5 inches wide), then this exposure chamber would be as short as 3.75 feet.

For the surface decontamination of the packaged vials or syringe tubs, the exposure chamber is followed by three successive aeration chambers. Each of these chambers is designed to be evacuated and filled with fresh air every minute. The volume of these chambers is equal to the entrance chamber and each chamber has a dedicated vacuum pump. We have found that the NO₂ concentration is reduced in proportion to the reduction in pressure. For example, reducing the pressure by 90% (going from 760 mmHg initial pressure to 76 mmHg final pressure) and then filling with fresh air will result in a 90% reduction in NO₂ concentration. Three successive aeration chambers, each providing a 90% reduction in concentration will result in a 99.9% reduction in NO₂. Where the exposure concentration is in the range of about 5000 ppm, the resulting concentration after transiting the aeration chambers would be about 5 ppm. It is quite feasible to reach 3% of the starting pressure in the aeration chambers during the time available, which would result in a final NO₂ concentration of 1.7 ppm. This level is well below the OSHA limit for the NO₂ gas.

Where the desired exposure concentration is 0.56% (10 mg/L), and this amount is filled into the entrance chamber every minute, then this chamber will receive 750 mg of NO₂ per minute (assuming a 75 liter chamber). This equates to 45 grams of sterilant per hour. For this example where 6 tubs are processed each minute, and each tub holds 100 syringes, then 36,000 syringes will be processed each hour. In 24 hours of operation, this system will use 1 kilogram of sterilant and 5 kilograms of scrubber medium, while processing 860,000 syringes.

### EXAMPLE 3

The in-line system may also be used to process filled syringes. Generally, after filling and capping with the stopper (with or without the plunger) in a sterile enclosure, the filled syringes are removed from the sterile filling enclosure and moved to a packaging location. The packaging is done outside of aseptic containment. Therefore, once the filled syringes are packaged, they must undergo a surface decontamination process. This may be needed for two reasons. First, there is a risk that the inner surface of the barrel may be unsterile because the filled syringe was open to the atmosphere (and thus, uncontrolled and possibly contaminated) during transfer to the packaging location. Stopper movement caused by pressure variations (as can occur during transportation) may cause expansion of the air bubble in the syringe, thereby exposing the syringe contents to an unsterile portion of the syringe barrel surface. Second, some prefilled syringes are used in an intraoperative setting, where the syringes are brought into the sterile field of the operating room. In both cases, the packaged prefilled syringes must be subjected to a sterilization (surface decontamination) procedure. In this case, the sterilization procedure must limit the vacuum levels to a range that is near (within 100 mmHg of) atmospheric pressure so that stopper movement does not occur or is sufficiently minimized. Adequate transfer of sterilant gas into and out of the package can occur with several rapid cycles of pressure reduction and gas addition to the chambers of this in-line system.

Although the invention has been described in detail for the purpose of illustration based on what are currently considered to be the most practical and preferred embodiments, it is to be understood that such detail is solely for that purpose and that the inventions are not limited to the disclosed embodiments, but, on the contrary, are intended to cover modifications and equivalent arrangements that are within the spirit and scope of the described embodiments. For example, it is to be understood that the present invention contemplates that, to the extent possible, one or more features of any embodiment can be combined with one or more features of any other embodiment.

1. A sterilization system, comprising:
a plurality of chambers, each chamber being constructed and arranged to be sealable in a gas impermeable configuration;
pairs of adjacent chambers being separated by doors configured to be movable between an open position in which gas may flow freely between the adjacent chambers and a closed position in which gas flow between the adjacent chambers is prevented;
a conveyor system, configured and arranged to convey objects between the chambers; and
at least one source of sterilant gas, communicated with at least one of the chambers to deliver a controlled amount of sterilant gas to the chamber, wherein, the chambers are arrayed end to end such that, in operation, an object to be sterilized placed on the conveyor system may be conveyed from an inlet end to an outlet end, passing through the at least one chamber communicated with the source of sterliant gas.

2. A sterilization system as in claim 1, further comprising a vacuum source, communicated with at least one of the chambers and configured and arranged to evacuate gases therefrom.

3. A sterilization system as in claim 1, wherein the doors provide a seal between chambers sufficiently gas tight that a pair of adjacent chambers may comprise an evacuated chamber and a chamber at ambient pressure, without substantial gas flow between the chambers.

4. A sterilization system as in claim 1, further comprising a controller, configured and arranged to control the conveyor system and the doors such that the object to be sterilized may be conveyed through the system during a sterilization operation in which evacuation, dwell and purge steps are executed.

5. A sterilization system as in claim 4, wherein the conveyor system and doors are operable such that while a first object to be sterilized undergoes a dwell step of a sterilization operation, a second object to be sterilized undergoes an evacuation step or a purge step.

6. A sterilization system as in claim 5, wherein the evacuation step or purge step consists of more than one lowering and raising of the chamber pressure.

7. A sterilization system as in claim 1, wherein the plurality of chambers comprises common mating structure such that each chamber may be selectively attached to each other chamber in a plurality of gas impermeable configurations.

8. A sterilization system as in claim in claim 1, wherein at least one chamber is configured and arranged to accept a gas input line and at least one other chamber is configured and arranged without any gas input.

9. A sterilization system as in claim 8, wherein the chamber configured and arranged without any gas input is longer than the chamber configured and arranged to accept the gas input line.

10. A sterilization system as in claim 1, wherein the source of sterilant gas is a source of NO₂.

11. A method of sterilizing an object, comprising:
sealing the object in a gas impermeable exposure chamber adjacent to a dwell chamber;
providing a selected amount of sterilant gas to the exposure chamber;
opening a passage between the exposure chamber and the dwell chamber;
conveying the object from the exposure chamber to the dwell chamber;
allowing the object to dwell within the dwell chamber during a selected dwell time;
opening a passage between the dwell chamber and an adjacent purge chamber; and
purging the object.

12. A method as in claim 11, wherein the sterilant gas is NO₂.

13. A method as in claim 11, further comprising two subsequent purge operations.

14. A method as in claim 11 wherein the allowing the object to dwell further comprises conveying the object within the dwell chamber during the selected dwell time,

15. A method as in claim 11, further comprising:
exposing the object in an additional exposure chamber; and
allowing the object to dwell in an additional dwell chamber.

16. A method as in claim 11, further comprising continuously conveying the object along a conveyor path.
